(19) United States
(12) Patent Application Publication
(10) Pub. No.: US 2026/0076624 A1
(43) Pub. Date: Mar. 19, 2026

(54) Biometric Handcuff Device

(71) Applicant: Sarah Schooley, Fairfield, OH (US)

(72) Inventor: Sarah Schooley, Fairfield, OH (US)

(21) Appl. No.: 19/089,671

(22) Filed: Mar. 25, 2025

Related U.S. Application Data
(60) Provisional application No. 63/696,522, filed on Sep. 19, 2024.

(51) Int. Cl. A61B 5/00; A61B 5/0205; E05B 75/00

(52) U.S. Cl. CPC A61B 5/6824; A61B 5/0002; A61B 5/0205; A61B 5/7282; A61B 5/7405; A61B 5/746; E05B 75/00

(57) ABSTRACT

A biometric handcuff device is disclosed, which identifies medical emergencies for those who are detained by police or security. The biometric handcuff device comprises a pair of law enforcement handcuffs that include a biometric sleeve inside the cuffs. The sleeve is constructed as a rubber insert that permanently stays in place within the cuffs. The biometric sleeve allows officers to track the breathing, blood pressure, and pulse of a handcuffed individual. The measurements taken may be observed remotely by police or medical personnel.

### CROSS-REFERENCE TO RELATED APPLICATION

The present application claims priority to, and the benefit of, U.S. Provisional Application No. 63/696,522, which was filed on Sep. 19, 2024, and is incorporated herein by reference in its entirety.

### FIELD OF THE INVENTION

The present invention relates generally to the field of biometric handcuff devices. More specifically, the present invention relates to specialized handcuffs that can help save lives of those who are detained by police or security. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in biometric handcuff devices. Generally, handcuffs are commonly used by law enforcement, security, military, and others to confine a perpetrator. Officers may not know how to recognize the early signs of a person needing medical assistance. Some officers may abuse their authority and hurt a perpetrator and then not report the true story to their supervisor.

Physical restraints, such as handcuffs or other bindings, are often used by law enforcement officials to restrain a detained individual. Often, these devices cover areas on an individual's body that are suited to measuring vitals readings, such as handcuffs covering a wearer's wrists. Currently, prior to transporting a restrained individual, law enforcement officials are trained to manually take vital readings and/or wait for emergency medical service providers to arrive for assessment when a restrained individual shows signs of a medical issue. Additionally, even with proper training, it may be difficult for a law enforcement official to assess and constantly monitor the physical condition of a restrained individual. This is especially true when the law enforcement official is transporting a restrained individual who did not present signs of a medical issue prior the law enforcement official placing the restrained individual in the vehicle. Thus, there is a long-felt need for a device which can simultaneously restrain an individual, monitor that individual's vital signs, and provide data and/or alerts to the law enforcement official for emerging or present medical issues.

Accordingly, there is a demand for an improved biometric handcuff device that features a biometric sleeve inside the handcuffs that tracks vitals like pulse, blood pressure, oxygen, etc. More particularly, there is a demand for a biometric handcuff device that prevents officers from being unaware of a handcuffed individual suffering from medical issues.

Therefore, there exists a long felt need in the art for a biometric handcuff device that provides users with specialized handcuffs that can help save lives of those who are detained by police or security. There is also a long felt need in the art for a biometric handcuff device that features a biometric sleeve inside the handcuffs that tracks vitals like pulse, blood pressure, oxygen, etc. Further, there is a long felt need in the art for a biometric handcuff device that prevents officers from being unaware of a handcuffed individual suffering from medical issues and needing emergency attention. Moreover, there is a long felt need in the art for a device that helps several institutions that utilize handcuffs like police, security officers, jail officers, and more. Further, there is a long felt need in the art for a biometric handcuff device that identifies medical emergencies for those who are detained by police or security. Finally, there is a long felt need in the art for a biometric handcuff device that allows remote observation of measurements by police or medical personnel.

The subject matter disclosed and claimed herein, in one embodiment thereof, comprises a biometric handcuff device. The device identifies medical emergencies for those who are detained by police or security. The biometric handcuff device comprises a pair of law enforcement handcuffs that include a biometric sleeve inside the cuffs. The sleeve is constructed as a rubber insert that permanently stays in place within the cuffs. The biometric sleeve allows officers to track the breathing, blood pressure, and pulse of a handcuffed individual. The measurements taken may be observed remotely by police or medical personnel.

In this manner, the biometric handcuff device of the present invention accomplishes all of the forgoing objectives and provides users with a biometric sleeve inside the handcuffs to track vitals. The device identifies medical emergencies. The device is constructed of a rubber insert.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a biometric handcuff device. The device identifies medical emergencies for those who are detained by police or security. The biometric handcuff device comprises a pair of law enforcement handcuffs that include a biometric sleeve inside the cuffs. The biometric sleeve allows officers to track the breathing, blood pressure, and pulse of a handcuffed individual. The measurements taken may be observed remotely by police or medical personnel.

In one embodiment, the biometric handcuff device monitors the biometrics of a handcuffed user. Further, the device not only monitors the biometrics, but also sends alerts to law enforcement officials or medical personnel of emerging or present medical issues. Thus, the biometric handcuff device helps several institutions that utilize handcuffs to monitor and track biometrics or vitals of those being detained.

In one embodiment, the biometric handcuff device is utilized with a pair of law enforcement handcuffs. The handcuffs are conventional handcuffs as is known in the art, which are typically used by police officers, security officers, jail officers, prison officers, etc., or any other suitable individual as is known in the art. Typically, the handcuffs comprise one cuff portion secured to another cuff portion via a connection portion, that can be a linked metal chain, a cable, a cord, or other suitable connector, etc.

In one embodiment, the biometric handcuff device comprises a housing sleeve that encompasses and houses a plurality of sensors for monitoring a user's biometrics or vitals. Typically, the housing sleeve is secured within an interior area of one of the cuff portions of the handcuffs, such that it makes contact with a user's wrists while the pair of handcuffs are in a locked position. The housing sleeve is secured via any suitable securing means as is known in the art, such as adhesives, glue, etc. Specifically, the housing sleeve is secured in such a way, that allows normal operation of the set of handcuffs (i.e., the normal open and close motions), and allows the pair of handcuffs to retain the ability to rotate its ratchet system during use to lock and unlock the handcuffs.

In one embodiment, the housing sleeve is typically constructed as a rubber insert that permanently stay in place within the handcuffs. The rubber insert allows the housing sleeve to be durable and strong, resisting damage to the device during use.

In one embodiment, the housing sleeve comprises a plurality of sensors for monitoring a user's biometrics, as well as a processor, a transceiver/receiver, a memory, a speaker, a battery, and a wireless communications module, as well as any circuitry needed for the device to fully function.

In one embodiment, the processor comprises various executable programs used to control the plurality of sensors, as well as the additional programs which calculate biometric data based on the data received from the plurality of sensors, and other suitable executables which are necessary for the function of the device. The processor and transceiver/receiver sends instructions to, receives data from, and processes outputs for the plurality of sensors of the device, as well.

In one embodiment, the processor calculates the user's biometrics by inputting the data from the sensors into corresponding algorithms, which are then sent via the transceiver/receiver and the wireless communications module to a paired smart device or computer for a law enforcement official to observe the biometric measurements. Further, the user's biometrics can be sent to a third party and not to law enforcements to observe the biometric measurements, such that the measurements and information are not tampered with. The paired smart device or computer remotely displays the user's biometrics and/or alerts law enforcement officials of an emerging or active medical condition of the user via sounding an audio alert through the speaker based on predetermined thresholds that can be programmed by law enforcement officials. Specifically, the wireless communications module utilizes Bluetooth, cellular, etc., or other wireless protocols to pair the device. Furthermore, a wireless signal is sent from the wireless communications module to a receiving smart device or computer belonging to the law enforcement official, whether on his or her person, or to a unit in his or her vehicle, or to a computer in a law enforcement office, etc.

In one embodiment, the plurality of sensors are positioned, such that they protrude through the housing sleeve to contact a user's wrist and are not tightened to an undesirable and/or uncomfortable position on the user. The plurality of sensors include but are not limited to: a blood pressure sensor; an SpO2 sensor (i.e., pulse oximeter); an electrocardiogram (ECG) sensor for monitoring a user's heart; an electrodermal activity (EDA) sensor for monitoring stress levels; a respiration sensor for monitoring breathing; and a heart rate/pulse sensor; and any other suitable sensors as is known in the art, depending on the needs and/or wants of a user.

In one embodiment, the blood pressure sensor acts to measure a user's blood pressure via measuring both a systolic pressure and normal blood flow pressure to calculate a user's blood pressure to some degree of accuracy. Additionally, current technology and algorithms are utilized, as well. The SpO2 sensor (i.e., pulse oximeter) utilizes a plurality of nodes with at least one emitter and at least one detector to measure the amount of oxygen in the user's blood. The ECG sensor measures the small amount of electrical current produced by the action of the user's heart pumping blood through their veins. The EDA sensor measures the changes in electrical activity resulting from changes in the user's sweat glands to determine a user's stress level. The respiration sensor monitors and detects a user's breathing. Finally, the heart rate sensor measures and monitors a user's pulse. If any of these measurements determined from the plurality of sensors, are above or below a predetermined threshold, an audible alert is sounded from the speaker and sounded through the paired device(s). Users can program the thresholds for each sensor via the paired smart device.

In one embodiment, the housing sleeve comprises an on/off button, which is manually used to turn the device on and off, as needed.

In one embodiment, the housing sleeve and components may remain in a dormant state awaiting activation to an activated state via movement from the user and/or law enforcement official. The movement may be in the form of briefly shaking the device, etc. Further, the device can be activated via just opening the pair of handcuffs. Additionally, the device can turn off and/or go into sleep mode after approximately 30-60 seconds of non-use (i.e., where no pulse or oxygen is detected).

In one embodiment, the battery is located within the housing sleeve and provides power to the device's components. The battery can either be replaceable or rechargeable. If replaceable, the battery is removed and replaced, as needed with new batteries. If rechargeable, the housing sleeve further comprises a charge port, such as a USB charge port or other suitable charging port as is known in the art which can be plugged in to charge the battery.

In yet another embodiment, the biometric handcuff device comprises a plurality of indicia.

In yet another embodiment, a method of identifying medical emergencies for those detained in handcuffs is disclosed. The method includes the steps of providing a biometric handcuff device comprising a pair of handcuffs with a biometric sleeve. The method also comprises securing the biometric sleeve to an inside of the handcuffs. Further, the method comprises pairing the biometric sleeve with a computer or smart device. The method also comprises securing the pair of handcuffs around a user's wrists. Finally, the method comprises tracking the biometrics, such as breathing, blood pressure and pulse of a handcuffed user.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the biometric handcuff device of the present invention showing a pair of law enforcement handcuffs in accordance with the disclosed architecture;

FIG. 2 illustrates a perspective view of one embodiment of the biometric handcuff device of the present invention showing the biometric sensor positioned within the handcuffs in accordance with the disclosed architecture;

FIG. 3 illustrates a perspective view of one embodiment of the biometric handcuff device of the present invention showing the handcuffs applied to an individual in accordance with the disclosed architecture;

FIG. 4 illustrates a perspective view of one embodiment of the biometric handcuff device of the present invention showing biometric readings displayed on a paired computer in accordance with the disclosed architecture;

FIG. 5 illustrates a perspective view of one embodiment of the biometric handcuff device of the present invention showing the device emitting an audible sound when normal health parameters are breached in accordance with the disclosed architecture; and

FIG. 6 illustrates a flowchart showing the method of identifying medical emergencies for those detained in handcuffs in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a biometric handcuff device that provides users with specialized handcuffs that can help save lives of those who are detained by police or security. There is also a long felt need in the art for a biometric handcuff device that features a biometric sleeve inside the handcuffs that tracks vitals like pulse, blood pressure, oxygen, etc. Further, there is a long felt need in the art for a biometric handcuff device that prevents officers from being unaware of a handcuffed individual suffering from medical issues and needing emergency attention. Moreover, there is a long felt need in the art for a device that helps several institutions that utilize handcuffs like police, security officers, jail officers, and more. Further, there is a long felt need in the art for a biometric handcuff device that identifies medical emergencies for those who are detained by police or security. Finally, there is a long felt need in the art for a biometric handcuff device that allows remote observation of measurements by police or medical personnel.

The present invention, in one exemplary embodiment, is a novel biometric handcuff device. The biometric handcuff device comprises a pair of law enforcement handcuffs that include a biometric sleeve inside the cuffs. The sleeve is constructed as a rubber insert that permanently stays in place within the cuffs. The biometric sleeve allows officers to track the breathing, blood pressure, and pulse of a handcuffed individual. The measurements taken may be observed remotely by police or medical personnel. The present invention also includes a novel method of identifying medical emergencies for those detained in handcuffs. The method includes the steps of providing a biometric handcuff device comprising a pair of handcuffs with a biometric sleeve. The method also comprises securing the biometric sleeve to an inside of the handcuffs. Further, the method comprises pairing the biometric sleeve with a computer or smart device. The method also comprises securing the pair of handcuffs around a user's wrists. Finally, the method comprises tracking the biometrics, such as breathing, blood pressure and pulse of a handcuffed user.

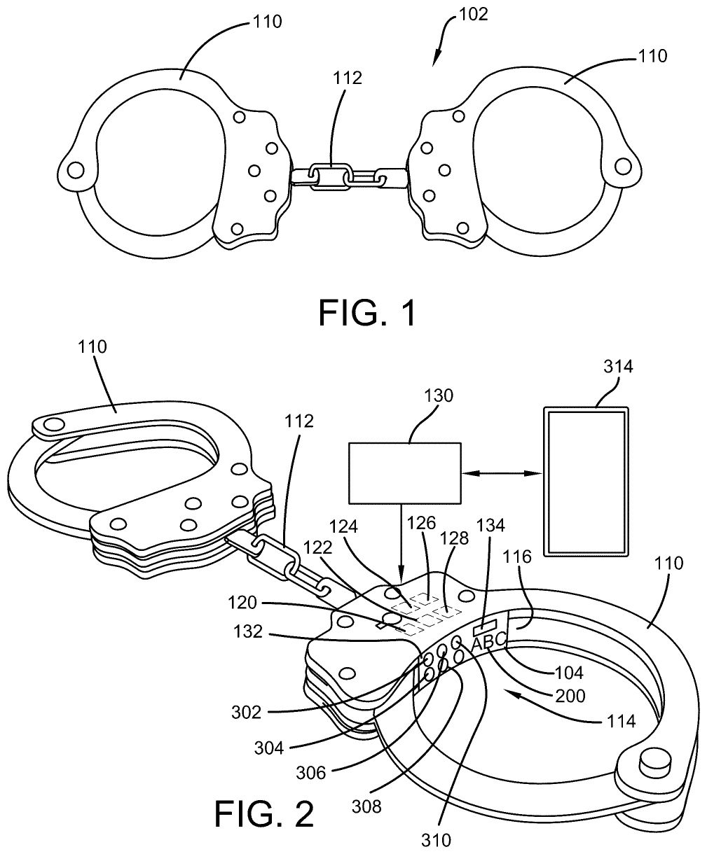

Referring initially to the drawings, FIG. 1 illustrates a perspective view of one embodiment of the biometric handcuff device 100 of the present invention. In the present embodiment, the biometric handcuff device 100 is an improved biometric handcuff device 100 that provides an enforcement official 108 with a means to track the biometrics of a handcuffed user 106. Specifically, the biometric handcuff device 100 comprises a pair of law enforcement handcuffs 102 and an attached biometric housing sleeve 104. The biometric housing sleeve 104 allows officers 108 to track the breathing, blood pressure, and pulse of a handcuffed individual/user 106. The measurements taken may be observed remotely by police 108 or medical personnel.

Generally, the biometric handcuff device 100 monitors the biometrics of a handcuffed user 106. Further, the device 100 not only monitors the biometrics, but also sends alerts to law enforcement officials 108 or medical personnel of emerging or present medical issues. Thus, the biometric handcuff device 100 helps several institutions that utilize handcuffs 102 to monitor and track biometrics or vitals of those being detained.

Further, the biometric handcuff device 100 is utilized with a pair of law enforcement handcuffs 102. The handcuffs 102 are conventional handcuffs as is known in the art, which are typically used by police officers 108, security officers, jail officers, prison officers, etc., or any other suitable individual as is known in the art. Typically, the handcuffs 102 comprise one cuff portion 110 secured to another cuff portion 110 via a connection portion 112, that can be a linked metal chain, a cable, a cord, or other suitable connector, etc.

As shown in FIG. 2, the biometric handcuff device 100 comprises a housing sleeve 104 that encompasses and houses a plurality of sensors 114 for monitoring a user's biometrics or vitals. Typically, the housing sleeve 104 is secured within an interior area 116 of one of the cuff portions 110 of the handcuffs 102, such that it makes contact with a user's wrists 118 while the pair of handcuffs 102 are in a locked position. The housing sleeve 104 is secured via any suitable securing means as is known in the art, such as adhesives, glue, etc. Specifically, the housing sleeve 104 is secured in such a way, that allows normal operation of the set of handcuffs 102 (i.e., the normal open and close motions), and allows the pair of handcuffs 102 to retain the ability to rotate its ratchet system during use to lock and unlock the handcuffs 102.

Further, the housing sleeve 104 is typically constructed as a rubber insert that permanently stays in place within the handcuffs 102. The rubber insert allows the housing sleeve 104 to be durable and strong, resisting damage to the device 100 during use. Typically, the housing sleeve 104 comprises an on/off button 132, which is manually used to turn the device 100 on and off, as needed. In another embodiment, the housing sleeve 104 and components may remain in a dormant state awaiting activation to an activated state via movement from the user 106 and/or law enforcement official 108. The movement may be in the form of briefly shaking the device 100, etc. Further, the device 100 can be activated via just opening the pair of handcuffs 102. Additionally, the device 100 can turn off and/or go into sleep mode after approximately 30-60 seconds of non-use (i.e., where no pulse or oxygen is detected).

Additionally, the housing sleeve 104 comprises a plurality of sensors 114 for monitoring a user's biometrics, as well as a processor 120, a transceiver/receiver 122, a memory 124, a speaker 126, a battery 128, and a wireless communications module 130, as well as any circuitry needed for the device 100 to fully function.

Furthermore, the battery 128 is located within the housing sleeve 104 and provides power to the device's components. The battery 128 can either be replaceable or rechargeable. If replaceable, the battery 128 is removed and replaced, as needed with new batteries. If rechargeable, the housing sleeve 104 further comprises a charge port 134, such as a USB charge port or other suitable charging port as is known in the art which can be plugged in to charge the battery 128.

The battery 128 may be a disposable battery or a rechargeable battery in the form of an alkaline, nickel-cadmium, nickel-metal hydride battery, etc., such as any 3V-12 volts DC battery or other conventional battery, such as A, AA, AAA, etc., that supplies power to the biometric handcuff device 100. Throughout this specification, the term “battery” may be used interchangeably to refer to one or more wet or dry cells or batteries of cells in which chemical energy is converted into electricity and used as a source of DC power. References to recharging or replacing the battery 128 may refer to recharging or replacing individual cells, individual batteries of cells, or a package of multiple battery cells as is appropriate for any given battery technology that may be used. In addition, a rechargeable embodiment of the battery 128 may be recharged using a USB port 134, wherein the USB port is a USB-A, USB-B, Micro-B, Micro-USB, Mini-USB, or USB-C port, etc.

In yet another embodiment, the biometric handcuff device 100 comprises a plurality of indicia 200. The housing sleeve 104 of the device 100 may include advertising, a trademark, or other letters, designs, or characters, printed, painted, stamped, or integrated into the housing sleeve 104, or any other indicia 200 as is known in the art. Specifically, any suitable indicia 200 as is known in the art can be included, such as but not limited to, patterns, logos, emblems, images, symbols, designs, letters, words, characters, animals, advertisements, brands, etc., that may or may not be handcuff, biometrics, or brand related.

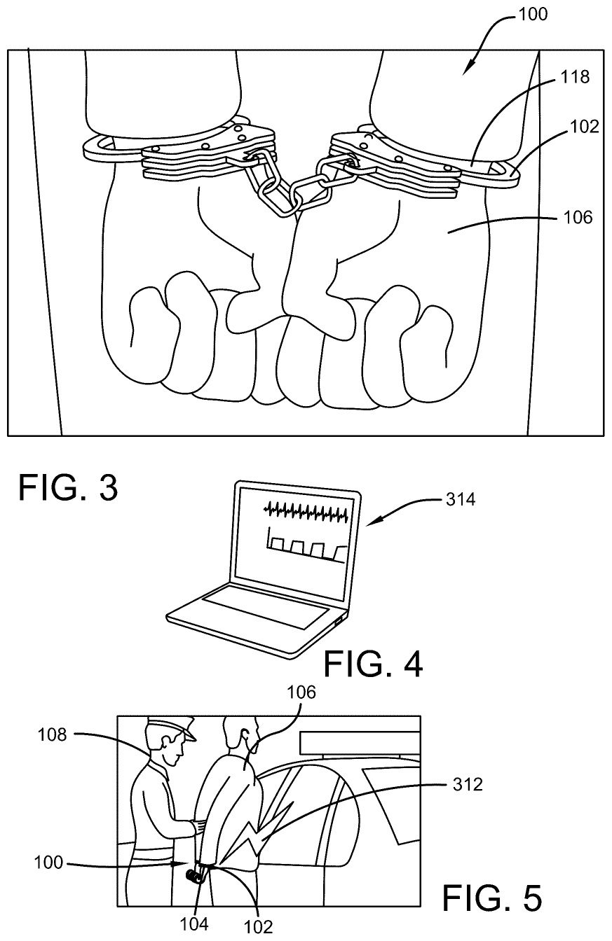

As shown in FIG. 3, the plurality of sensors 114 are positioned, such that they protrude through the housing sleeve 104 to contact a user's wrist 118 and are not tightened to an undesirable and/or uncomfortable position on the user 106. The plurality of sensors 114 include but are not limited to: a blood pressure sensor 300; an SpO2 sensor (i.e., pulse oximeter) 302; an electrocardiogram (ECG) sensor 304 for monitoring a user's heart; an electrodermal activity (EDA) sensor 306 for monitoring stress levels; a respiration sensor 308 for monitoring breathing; and a heart rate/pulse sensor 310; and any other suitable sensors as is known in the art, depending on the needs and/or wants of a user 106 or law enforcement official 108.

Furthermore, the blood pressure sensor 300 acts to measure a user's blood pressure via measuring both a systolic pressure and normal blood flow pressure to calculate a user's blood pressure to some degree of accuracy. Additionally, current technology and algorithms are utilized, as well. The SpO2 sensor (i.e., pulse oximeter) 302 utilizes a plurality of nodes with at least one emitter and at least one detector to measure the amount of oxygen in the user's blood. The ECG sensor 304 measures the small amount of electrical current produced by the action of the user's heart pumping blood through their veins. The EDA sensor 306 measures the changes in electrical activity resulting from changes in the user's sweat glands to determine a user's stress level. The respiration sensor 308 monitors and detects a user's breathing. Finally, the heart rate sensor 310 measures and monitors a user's pulse. If any of these measurements determined from the plurality of sensors 114, are above or below a predetermined threshold, an audible alert 312 is sounded from the speaker 126 and sounded through the paired device(s) 314. Users (i.e., law enforcement officials 108) can program the thresholds for each sensor 114 via the paired smart device 314.

As shown in FIG. 4, the processor 120 comprises various executable programs used to control the plurality of sensors 114, as well as the additional programs which calculate biometric data based on the data received from the plurality of sensors 114, and other suitable executables which are necessary for the function of the device 100. The processor 120 and transceiver/receiver 122 sends instructions to, receives data from, and processes outputs for the plurality of sensors 114 of the device 100, as well.

As shown in FIG. 5, the processor 120 calculates the user's biometrics by inputting the data from the sensors 114 into corresponding algorithms, which are then sent via the transceiver/receiver 122 and the wireless communications module 130 to a paired smart device 314 or computer for a law enforcement official 108 to observe the biometric measurements. Further, the user's biometrics can be sent to a third party and not to law enforcements to observe the biometric measurements, such that the measurements and information are not tampered with. The paired smart device 314 or computer remotely displays the user's biometrics and/or alerts law enforcement officials 108 of an emerging or active medical condition of the user 106 via sounding an audio alert 312 through the speaker 126 based on predetermined thresholds that can be programmed by law enforcement officials 108. Specifically, the wireless communications module 130 utilizes any type of wireless communication, including, but not limited to, WIFI, BLUETOOTH, RFID, NFC, etc., to pair the device 100. Furthermore, a wireless signal is sent from the wireless communications module 130 to a receiving smart device 314 or computer belonging to the law enforcement official 108, whether on his or her person, or to a unit in his or her vehicle, or to a computer in a law enforcement office, etc. Once paired, a law enforcement official 108 could control the biometric handcuff device 100 via the paired device 314.

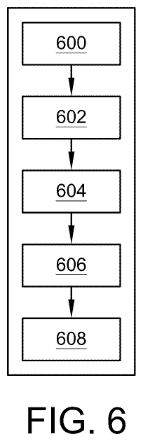

FIG. 6 illustrates a flowchart of the method of identifying medical emergencies for those detained in handcuffs. The method includes the steps of at 600, providing a biometric handcuff device comprising a pair of handcuffs with a biometric sleeve. The method also comprises at 602, securing the biometric sleeve to an inside of the handcuffs. Further, the method comprises at 604, pairing the biometric sleeve with a computer or smart device. The method also comprises at 606, securing the pair of handcuffs around a user's wrists. Finally, the method comprises at 608, tracking the biometrics, such as breathing, blood pressure and pulse of a handcuffed user.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “biometric handcuff device”, “biometric device”, “handcuff device”, and “device” are interchangeable and refer to the biometric handcuff device 100 of the present invention.

Notwithstanding the forgoing, the biometric handcuff device 100 of the present invention can be of any suitable size and configuration as is known in the art without affecting the overall concept of the invention, provided that it accomplishes the above stated objectives. One of ordinary skill in the art will appreciate that the biometric handcuff device 100 as shown in FIGS. 1-6 is for illustrative purposes only, and that many other sizes and shapes of the biometric handcuff device 100 are well within the scope of the present disclosure. Although the dimensions of the biometric handcuff device 100 are important design parameters for user convenience, the biometric handcuff device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A biometric handcuff device that provides a user with a means to track biometrics of a handcuffed user, the biometric handcuff device comprising:
a housing sleeve;
wherein the housing sleeve is secured to a pair of handcuffs; and
further wherein the housing sleeve comprises a plurality of sensors, a processor, a transceiver/receiver, and a memory to track biometrics of a handcuffed user.

2. The biometric handcuff device of claim 1, wherein the pair of handcuffs comprise one cuff portion secured to another cuff portion via a connection portion, wherein the housing sleeve is secured to an interior of one of the cuff portions such that it makes contact with a user's wrists while the pair of handcuffs are in a locked position.

3. The biometric handcuff device of claim 2, wherein the housing sleeve is secured via glue or adhesives.

4. The biometric handcuff device of claim 3, wherein the housing sleeve is constructed as a rubber insert that permanently stays in place within the pair of handcuffs.

5. The biometric handcuff device of claim 4, wherein the housing sleeve comprises an on/off button.

6. The biometric handcuff device of claim 5, wherein the housing sleeve comprises a replaceable battery.

7. The biometric handcuff device of claim 5, wherein the housing sleeve comprises a rechargeable battery and a charge port.

8. The biometric handcuff device of claim 5 further comprising a plurality of indicia.

9. The biometric handcuff device of claim 8, wherein the plurality of sensors are positioned, such that they protrude through the housing sleeve to contact a user's wrist.

10. The biometric handcuff device of claim 9, wherein the plurality of sensors include but are not limited to, a blood pressure sensor; an SpO2 sensor; an electrocardiogram (ECG) sensor; an electrodermal activity (EDA) sensor; a respiration sensor; and a heart rate sensor.

11. The biometric handcuff device of claim 10, wherein the processor calculates a user's biometrics by inputting data from the plurality of sensors into corresponding algorithms, which are then sent via the transceiver/receiver to send instructions to, receive data from, and process outputs for the plurality of sensors.

12. The biometric handcuff device of claim 11, wherein the housing sleeve comprises a wireless communications module to pair a smart device or computer to the biometric handcuff device, and wherein the paired smart device or computer remotely displays the user's biometrics.

13. The biometric handcuff device of claim 12, wherein the housing sleeve comprises a speaker, such that an audio alert can be sounded through the speaker to alert law enforcement officials of an emerging or active medical condition of the user.

14. A biometric handcuff device that provides a user with a means to track biometrics of a handcuffed user, the biometric handcuff device comprising:
a housing sleeve with an on/off button;
wherein the pair of handcuffs comprise one cuff portion secured to another cuff portion via a connection portion, wherein the housing sleeve is secured to an interior of one of the cuff portions such that it makes contact with a user's wrists while the pair of handcuffs are in a locked position;
wherein the housing sleeve comprises a plurality of sensors, a processor, a transceiver/receiver, and a memory to track biometrics of a handcuffed user;
wherein the plurality of sensors are positioned, such that they protrude through the housing sleeve to contact a user's wrist;
wherein the plurality of sensors include but are not limited to, a blood pressure sensor; an SpO2 sensor; an electrocardiogram (ECG) sensor; an electrodermal activity (EDA) sensor; a respiration sensor; and a heart rate sensor;
wherein the processor calculates a user's biometrics by inputting data from the plurality of sensors into corresponding algorithms, which are then sent via the transceiver/receiver to send instructions to, receive data from, and process outputs for the plurality of sensors; and
further wherein the housing sleeve comprises a wireless communications module to pair a smart device or computer to the biometric handcuff device, and wherein the paired smart device or computer remotely displays the user's biometrics.

15. The biometric handcuff device of claim 14, wherein the housing sleeve is secured via glue or adhesives.

16. The biometric handcuff device of claim 14, wherein the housing sleeve is constructed as a rubber insert that permanently stays in place within the pair of handcuffs.

17. The biometric handcuff device of claim 14, wherein the housing sleeve comprises a replaceable battery.

18. The biometric handcuff device of claim 14, wherein the housing sleeve comprises a rechargeable battery and a charge port.

19. The biometric handcuff device of claim 14, wherein the housing sleeve comprises a speaker, such that an audio alert can be sounded through the speaker to alert law enforcement officials of an emerging or active medical condition of the user.

20. A method of identifying medical emergencies for those detained in handcuffs, the method comprising the following steps:
providing a biometric handcuff device comprising a pair of handcuffs with a biometric sleeve;
securing the biometric sleeve to an inside of the handcuffs;
pairing the biometric sleeve with a computer or smart device;
securing the pair of handcuffs around a user's wrists; and
tracking the biometrics, such as breathing, blood pressure and pulse of a handcuffed user.
